(19) United States
(12) Patent Application Publication
(10) Pub. No.: US 2024/0266034 A1
(43) Pub. Date: Aug. 8, 2024

(54) INFORMATION PROCESSING APPARATUS, INFORMATION PROCESSING METHOD, AND INFORMATION PROCESSING PROGRAM

(71) Applicant: FUJIFILM Corporation, Tokyo (JP)

(72) Inventor: Yohei MOMOKI, Tokyo (JP)

(73) Assignee: FUJIFILM Corporation, Tokyo (JP)

(21) Appl. No.: 18/617,632

(22) Filed: Mar. 26, 2024

Related U.S. Application Data
(63) Continuation of application No. PCT/JP2022/036598, filed on Sep. 29, 2022.

(30) Foreign Application Priority Data
Sep. 30, 2021 (JP) 2021-162034

(51) Int. Cl. G16H 30/40; G06F 40/166; G16H 30/20

(52) U.S. Cl. CPC G16H 30/40; G06F 40/166; G16H 30/20

(57) ABSTRACT

An information processing apparatus including at least one processor, wherein the processor is configured to: generate a plurality of sentences based on a plurality of pieces of element information used for diagnosis; and correct, in a case where some element information among the plurality of pieces of element information is changed, only a first sentence corresponding to the element information to be changed among the plurality of sentences.

### CROSS REFERENCE TO RELATED APPLICATIONS

This application is a continuation of International Application No. PCT/JP2022/036598, filed on Sep. 29, 2022, which claims priority from Japanese Patent Application No. 2021-162034, filed on Sep. 30, 2021. The entire disclosure of each of the above applications is incorporated herein by reference.

### BACKGROUND

### Technical Field

The present disclosure relates to an information processing apparatus, an information processing method, and an information processing program.

### Related Art

In the related art, image diagnosis is performed using medical images obtained by imaging apparatuses such as computed tomography (CT) apparatuses and magnetic resonance imaging (MRI) apparatuses. In addition, image diagnosis is made by analyzing medical images via computer aided detection/diagnosis (CAD) using a discriminator in which learning is performed by deep learning or the like, and detecting and/or diagnosing regions of interest including structures, lesions, and the like included in the medical images. The medical images and analysis results via CAD are transmitted to a terminal of a healthcare professional such as a radiologist who interprets the medical images. The healthcare professional such as a radiologist interprets the medical image by referring to the medical image and analysis result using his or her own terminal and creates an interpretation report.

In addition, various methods have been proposed to support the creation of medical documents such as interpretation reports in order to reduce the burden of the interpretation work of a radiologist. For example, JP2019-153250A discloses a technology for creating a medical document such as an interpretation report based on a keyword input by a radiologist and an analysis result of a medical image. In the technology disclosed in JP2019-153250A, a sentence to be included in the interpretation report is created by using a recurrent neural network trained to generate a sentence from input characters.

For example, JP2020-123109A discloses a technology for reducing a load on creating a medical report by creating a template for the current medical report based on medical reports created in the past and displaying update targets that should be updated in the current report in an identifiable manner on a display.

In recent years, as the performance of imaging apparatuses has improved, the amount of information on analysis results obtained from medical images has tended to increase, and therefore the amount of sentences described in medical documents such as interpretation reports has also tended to increase. In this case, even in a case where a user corrects the analysis result obtained from the medical image, it may not be possible to immediately specify the sentence that has been corrected accordingly, making it difficult to check. Further, even in a case where the user corrects only a part of the analysis result obtained from the medical image, the entire sentence may be corrected, resulting in the user having to check the entire sentence again, which may be troublesome.

### SUMMARY

The present disclosure provides an information processing apparatus, an information processing method, and an information processing program capable of supporting creation of medical documents.

According to a first aspect of the present disclosure, there is provided an information processing apparatus comprising at least one processor, in which the processor may be configured to: generate a plurality of sentences based on a plurality of pieces of element information used for diagnosis; and correct, in a case where some element information among the plurality of pieces of element information is changed, only a first sentence corresponding to the element information to be changed among the plurality of sentences.

According to a second aspect of the present disclosure, in the first aspect, the processor may be configured to classify the plurality of sentences into the first sentence and a second sentence that does not correspond to the element information to be changed; and correct only the first sentence among the first sentence and the second sentence based on the changed element information.

According to a third aspect of the present disclosure, in the first or second aspect, the element information may be information indicating at least one of a name, a property, a measured value, a position, or an estimated disease name related to a region of interest included in a medical image, or an imaging method, an imaging condition, or an imaging date and time related to imaging of the medical image.

According to a fourth aspect of the present disclosure, in the third aspect, the region of interest may be at least one of a region of a structure included in the medical image or a region of an abnormal shadow included in the medical image.

According to a fifth aspect of the present disclosure, in any one of the first to fourth aspects, the processor may be configured to perform control to display the plurality of sentences on a display device.

According to a sixth aspect of the present disclosure, in the fifth aspect, the processor may be configured to perform control to display the plurality of sentences on the display device by grouping the plurality of sentences based on the element information to which the sentences correspond.

According to a seventh aspect of the present disclosure, in the fifth or sixth aspect, the processor may be configured to perform control to display the corrected first sentence in an emphasized manner on the display device.

According to an eighth aspect of the present disclosure, in any one of the fifth to seventh aspects, the processor may be configured to, in a case of receiving a change in the element information, perform control to display the first sentence before correction in an emphasized manner on the display device.

According to a ninth aspect of the present disclosure, in any one of the first to eighth aspects, the processor may be configured to: acquire a medical image; and generate the element information based on the acquired medical image.

According to a tenth aspect of the present disclosure, in any one of the first to ninth aspects, the information processing apparatus may further comprise an input unit, and the processor may be configured to generate the element information based on information input via the input unit.

According to an eleventh aspect of the present disclosure, in any one of the first to tenth aspects, the processor may be configured to acquire the element information from an external device.

According to a twelfth aspect of the present disclosure, there is provided an information processing method comprising: generating a plurality of sentences based on a plurality of pieces of element information used for diagnosis; and correcting, in a case where some element information among the plurality of pieces of element information is changed, only a first sentence corresponding to the element information to be changed among the plurality of sentences.

According to a thirteenth aspect of the present disclosure, there is provided an information processing program causing a computer to execute: generating a plurality of sentences based on a plurality of pieces of element information used for diagnosis; and correcting, in a case where some element information among the plurality of pieces of element information is changed, only a first sentence corresponding to the element information to be changed among the plurality of sentences.

The information processing apparatus, the information processing method, and the information processing program according to the aspects of the present disclosure can support the creation of medical documents.

### BRIEF DESCRIPTION OF THE DRAWINGS

FIG. 1 is a diagram showing an example of a schematic configuration of an information processing system.

FIG. 2 is a block diagram showing an example of a hardware configuration of an information processing apparatus.

FIG. 3 is a block diagram showing an example of a functional configuration of the information processing apparatus.

FIG. 4 is a diagram showing an example of a screen displayed on a display.

FIG. 5 is a diagram showing an example of a screen displayed on a display.

FIG. 6 is a diagram showing an example of a screen displayed on a display.

FIG. 7 is a flowchart showing an example of information processing.

FIG. 8 is a diagram showing an example of a screen displayed on a display.

FIG. 9 is a diagram showing an example of a graph structure.

### DETAILED DESCRIPTION

Hereinafter, an exemplary embodiment of the present disclosure will be described with reference to the drawings.

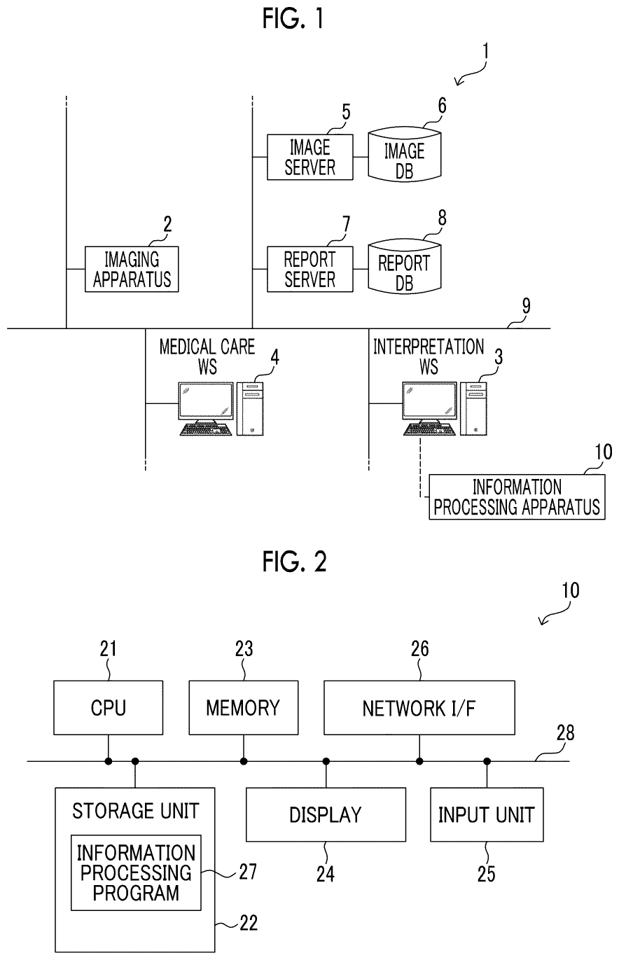

First, a configuration of an information processing system 1 to which an information processing apparatus of the present disclosure is applied will be described. FIG. 1 is a diagram showing a schematic configuration of the information processing system 1. The information processing system 1 shown in FIG. 1 performs imaging of an examination target part of a subject and storing of a medical image acquired by the imaging based on an examination order from a doctor in a medical department using a known ordering system. In addition, the information processing system 1 performs an interpretation work of a medical image and creation of an interpretation report by a radiologist and viewing of the interpretation report by a doctor of a medical department that is a request source.

As shown in FIG. 1, the information processing system 1 includes an imaging apparatus 2, an interpretation work station (WS) 3 that is an interpretation terminal, a medical care WS 4, an image server 5, an image database (DB) 6, a report server 7, and a report DB 8. The imaging apparatus 2, the interpretation WS 3, the medical care WS 4, the image server 5, the image DB 6, the report server 7, and the report DB 8 are connected to each other via a wired or wireless network 9 in a communicable state.

Each apparatus is a computer on which an application program for causing each apparatus to function as a component of the information processing system 1 is installed. The application program may be recorded on, for example, a recording medium, such as a digital versatile disc (DVD) or a compact disc read-only memory (CD-ROM), and distributed, and be installed on the computer from the recording medium. In addition, the application program may be stored in, for example, a storage apparatus of a server computer connected to the network 9 or in a network storage in a state in which it can be accessed from the outside, and be downloaded and installed on the computer in response to a request.

The imaging apparatus 2 is an apparatus (modality) that generates a medical image showing a diagnosis target part of the subject by imaging the diagnosis target part. Specifically, examples of the imaging apparatus include a simple X-ray imaging apparatus, a CT apparatus, an MRI apparatus, a positron emission tomography (PET) apparatus, and the like. The medical image generated by the imaging apparatus 2 is transmitted to the image server 5 and is stored in the image DB 6.

The interpretation WS 3 is a computer used by, for example, a healthcare professional such as a radiologist of a radiology department to interpret a medical image and to create an interpretation report, and encompasses an information processing apparatus 10 according to the present exemplary embodiment. In the interpretation WS 3, a viewing request for a medical image to the image server 5, various types of image processing for the medical image received from the image server 5, display of the medical image, and input reception of a sentence regarding the medical image are performed. In the interpretation WS 3, analysis processing for medical images, support for creating an interpretation report based on the analysis result, a registration request and a viewing request for the interpretation report to the report server 7, and display of the interpretation report received from the report server 7 are performed. The above processes are performed by the interpretation WS 3 executing software programs for respective processes.

The medical care WS 4 is a computer used by, for example, a healthcare professional such as a doctor in a medical department to observe a medical image in detail, view an interpretation report, create an electronic medical record, and the like, and is configured to include a processing device, a display device such as a display, and an input device such as a keyboard and a mouse. In the medical care WS 4, a viewing request for the medical image to the image server 5, display of the medical image received from the image server 5, a viewing request for the interpretation report to the report server 7, and display of the interpretation report received from the report server 7 are performed. The above processes are performed by the medical care WS 4 executing software programs for respective processes.

The image server 5 is a general-purpose computer on which a software program that provides a function of a database management system (DBMS) is installed. The image server 5 is connected to the image DB 6. The connection form between the image server 5 and the image DB 6 is not particularly limited, and may be a form connected by a data bus, or a form connected to each other via a network such as a network attached storage (NAS) and a storage area network (SAN).

The image DB 6 is realized by, for example, a storage medium such as a hard disk drive (HDD), a solid-state drive (SSD), and a flash memory. In the image DB 6, the medical image acquired by the imaging apparatus 2 and accessory information attached to the medical image are registered in association with each other.

The accessory information may include, for example, identification information such as an image identification (ID) for identifying a medical image, a tomographic ID assigned to each tomographic image included in the medical image, a subject ID for identifying a subject, and an examination ID for identifying an examination. In addition, the accessory information may include, for example, information related to imaging such as an imaging method, an imaging condition, and an imaging date and time related to imaging of a medical image. The “imaging method” and “imaging condition” are, for example, a type of the imaging apparatus 2, an imaging part, an imaging protocol, an imaging sequence, an imaging method, the presence or absence of use of a contrast medium, and the like. In addition, the accessory information may include information related to the subject such as the name, age, and gender of the subject.

In a case where the image server 5 receives a request to register a medical image from the imaging apparatus 2, the image server 5 prepares the medical image in a format for a database and registers the medical image in the image DB 6. In addition, in a case where the viewing request from the interpretation WS 3 and the medical care WS 4 is received, the image server 5 searches for a medical image registered in the image DB 6 and transmits the searched for medical image to the interpretation WS 3 and to the medical care WS 4 that are viewing request sources.

The report server 7 is a general-purpose computer on which a software program that provides a function of a database management system is installed. The report server 7 is connected to the report DB 8. The connection form between the report server 7 and the report DB 8 is not particularly limited, and may be a form connected by a data bus or a form connected via a network such as a NAS and a SAN.

The report DB 8 is realized by, for example, a storage medium such as an HDD, an SSD, and a flash memory. In the report DB 8, an interpretation report created in the interpretation WS 3 is registered.

Further, in a case where the report server 7 receives a request to register the interpretation report from the interpretation WS 3, the report server 7 prepares the interpretation report in a format for a database and registers the interpretation report in the report DB 8. Further, in a case where the report server 7 receives the viewing request for the interpretation report from the interpretation WS 3 and the medical care WS 4, the report server 7 searches for the interpretation report registered in the report DB 8, and transmits the searched for interpretation report to the interpretation WS 3 and to the medical care WS 4 that are viewing request sources.

The network 9 is, for example, a network such as a local area network (LAN) and a wide area network (WAN). The imaging apparatus 2, the interpretation WS 3, the medical care WS 4, the image server 5, the image DB 6, the report server 7, and the report DB 8 included in the information processing system 1 may be disposed in the same medical institution, or may be disposed in different medical institutions or the like. Further, the number of each apparatus of the imaging apparatus 2, the interpretation WS 3, the medical care WS 4, the image server 5, the image DB 6, the report server 7, and the report DB 8 is not limited to the number shown in FIG. 1, and each apparatus may be composed of a plurality of apparatuses having the same functions.

Next, the information processing apparatus 10 according to the present exemplary embodiment will be described. The information processing apparatus 10 has a function of supporting the creation of a medical document such as an interpretation report based on a medical image captured by the imaging apparatus 2. As described above, the information processing apparatus 10 is encompassed in the interpretation WS 3.

First, with reference to FIG. 2, an example of a hardware configuration of the information processing apparatus 10 according to the present exemplary embodiment will be described. As shown in FIG. 2, the information processing apparatus 10 includes a central processing unit (CPU) 21, a non-volatile storage unit 22, and a memory 23 as a temporary storage area. Further, the information processing apparatus 10 includes a display 24 such as a liquid crystal display, an input unit 25 such as a keyboard and a mouse, and a network interface (I/F) 26. The network I/F 26 is connected to the network 9 and performs wired or wireless communication. The CPU 21, the storage unit 22, the memory 23, the display 24, the input unit 25, and the network I/F 26 are connected to each other via a bus 28 such as a system bus and a control bus so that various types of information can be exchanged. The display 24 is an example of a display device of the present disclosure.

The storage unit 22 is realized by, for example, a storage medium such as an HDD, an SSD, and a flash memory. An information processing program 27 in the information processing apparatus 10 is stored in the storage unit 22. The CPU 21 reads out the information processing program 27 from the storage unit 22, loads the read-out program into the memory 23, and executes the loaded information processing program 27. The CPU 21 is an example of a processor of the present disclosure. As the information processing apparatus 10, for example, a personal computer, a server computer, a smartphone, a tablet terminal, a wearable terminal, or the like can be appropriately applied.

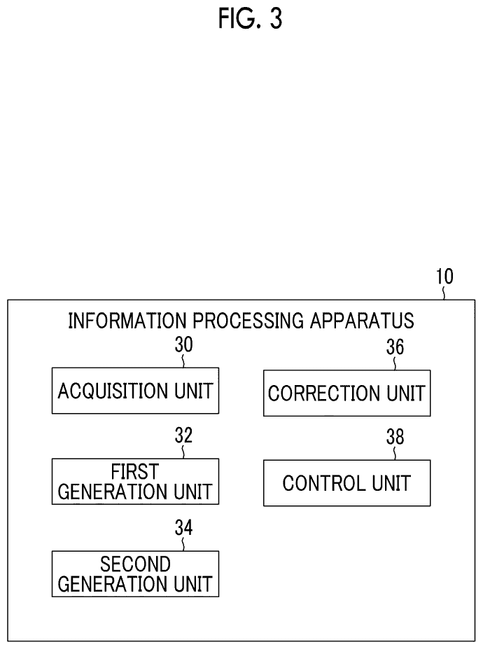

Next, with reference to FIG. 3, an example of a functional configuration of the information processing apparatus 10 according to the present exemplary embodiment will be described. As shown in FIG. 3, the information processing apparatus 10 includes an acquisition unit 30, a first generation unit 32, a second generation unit 34, a correction unit 36, and a control unit 38. In a case where the CPU 21 executes the information processing program 27, the CPU 21 functions as the acquisition unit 30, the first generation unit 32, the second generation unit 34, the correction unit 36, and the control unit 38.

The acquisition unit 30 acquires a medical image to be created as an interpretation report from the image server 5. The medical image is an example of an image of the present disclosure. In the following description, an example will be described in which the medical image acquired by the acquisition unit 30 is a medical image related to lungs.

The first generation unit 32 generates element information used for diagnosis based on the medical image acquired by the acquisition unit 30. Specifically, the first generation unit 32 extracts a region of interest including at least one of a region of a structure (for example, organs, tissues, and the like) included in the medical image or a region of an abnormal shadow (for example, the shadow due to a lesion such as a nodule) included in the medical image. For the extraction of the region of interest, for example, a trained model such as a convolutional neural network (CNN), which has been trained in advance to input a medical image and output a region of interest extracted from the medical image, may be used. Further, the first generation unit 32 may extract a region in the medical image designated by a user via the input unit 25 as a region of interest.

Further, the first generation unit 32 generates element information related to the region of interest extracted from the medical image. For the generation of the element information by the first generation unit 32, for example, a trained model such as a CNN, which has been trained in advance to input a region of interest in the medical image and output element information related to the region of interest, may be used.

Here, examples of the element information include information indicating at least one of a name (type), a property, a measured value, a position, or an estimated disease name (including a negative or positive evaluation result) related to a region of interest included in a medical image. Examples of names (types) include the names of structures such as “lung field”, “bronchus”, and “pleura”, and the names of abnormal shadows such as “nodule”, “cavity”, and “calcification”. The property mainly refers to the feature of the abnormal shadow, and examples thereof include findings indicating opacity such as “solid” and “ground-glass”, margin shapes such as “well-defined/ill-defined”, “smooth/irregular”, “spiculated”, “lobulated”, and “lagged”, and an overall shape such as “round” and “irregular form”. Further, for example, findings qualitatively indicating the size and amount of abnormal shadows (“large/small”, “single/multiple”, and the like), and findings regarding the presence or absence of contrast enhancement, washout, and the like may be used.

The measured value is a value that can be quantitatively measured from a medical image, and examples thereof include, for example, a max diameter, a CT value whose unit is HU, the number of regions of interest in a case where there are a plurality of regions of interest, and a distance between regions of interest. The position refers to a position in an image regarding a region of interest or a positional relationship with another region of interest, and examples thereof include “internal area”, “marginal area”, “around area”, and “local area”. The estimated disease name is an evaluation result estimated by the first generation unit 32 based on the abnormal shadow, and examples thereof include the disease name such as “cancer” and “inflammation” and the evaluation result such as “negative/positive” regarding each property, and the like.

Further, as described above, each medical image is attached by accessory information including information related to imaging at the time of being registered in the image DB 6. Therefore, the first generation unit 32 may generate, as element information, information indicating at least one of an imaging method, an imaging condition, or an imaging date and time related to the imaging of the medical image based on the accessory information attached to the medical image acquired by the acquisition unit 30 from the image server 5. Further, for example, the first generation unit 32 may acquire information included in an examination order and an electronic medical record, information indicating various test results such as a blood test and an infectious disease test, information indicating the result of a health diagnosis, and the like from the external device such as the medical care WS 4, and generate the acquired information as element information as appropriate.

In addition, the first generation unit 32 may generate element information based on the information input via the input unit 25. For example, the first generation unit 32 may generate element information based on the keywords input by the user via the input unit 25. Further, for example, the first generation unit 32 may present a candidate for element information on the display 24 and receive the designation of the element information by the user.

The second generation unit 34 generates a plurality of sentences based on a plurality of pieces of element information generated by the first generation unit 32 and used for diagnosis. Specifically, the second generation unit 34 may generate a sentence by inputting the element information generated by the first generation unit 32 to a trained model such as a CNN, which has been trained in advance such that the input is element information and the output is a sentence.

Incidentally, in an interpretation report regarding a medical image, there are cases where rules such as an agreement within a medical institution or a user's preference are established regarding the description order of element information in the sentence. For example, in a case of describing an abnormal shadow, it may be desired to describe the findings such as position, size, and overall shape first, and detailed findings of the marginal area and the internal area later. Further, for example, it may be desired to describe the malignant findings first and the benign findings later. Further, for example, in a case of describing the comparison result with the past medical image, it may be desired to describe the changed portion first and the unchanged portion later.

Thus, it is preferable that the second generation unit 34 generates a plurality of sentences by reflecting the above rules. For example, in a case of generating a plurality of sentences using a trained model, by reflecting the rules in advance in supervised training data used in the learning phase of the model, it is possible to obtain a trained model that reflects the rules.

Further, for example, the second generation unit 34 may group a plurality of pieces of element information according to predetermined rules, and input the element information to the trained model for each group, thereby generating sentences for each group.

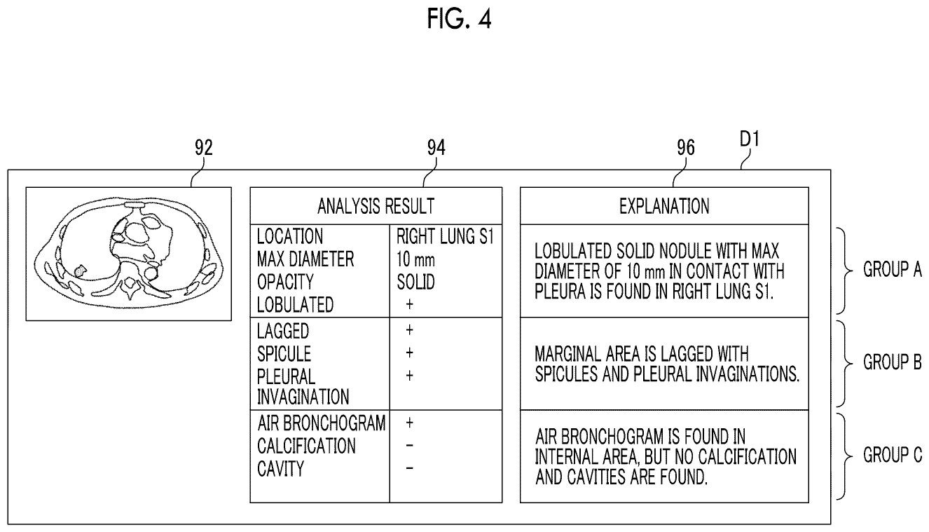

The control unit 38 performs control to display the plurality of sentences generated by the second generation unit 34 on the display 24. FIG. 4 shows an example of a screen D1 displayed on the display 24 by the control unit 38. As shown in FIG. 4, the screen D1 includes a region 92 where the medical image acquired by the acquisition unit 30 is displayed, a region 94 where the element information generated by the first generation unit 32 is displayed, and a region 96 where the plurality of sentences generated by the second generation unit 34 are displayed.

Further, as shown in FIG. 4, the control unit 38 may perform control to display the plurality of sentences on the display 24 by grouping the plurality of sentences based on element information to which the sentences correspond. Grouping is performed in the same manner as the above-described rules regarding the description order of element information. In the example of FIG. 4, grouping is performed such that group A includes element information of high importance, group B includes element information regarding the marginal area, and group C includes element information regarding the internal area. By displaying the element information and sentences in groups in this way on the display 24, the visibility of the element information included in each sentence can be improved.

Further, the control unit 38 receives changes made by the user to the element information. This is because the element information generated by the first generation unit 32 may include, for example, misdiagnosis via CAD, information that the user determines to be description unnecessary, and information that the user desires to describe but has not been generated.

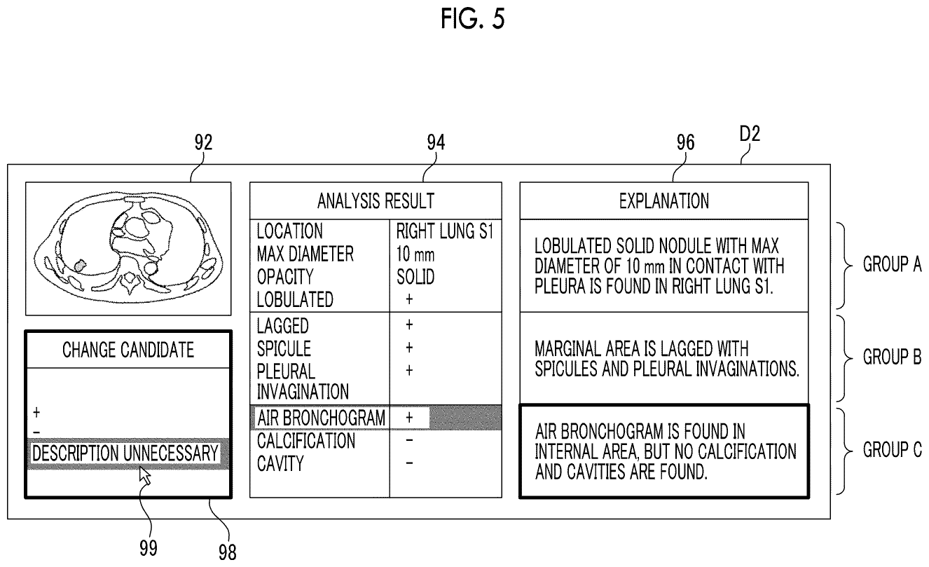

FIG. 5 shows an example of a screen D2 for receiving changes in element information, which is displayed on the display 24 by the control unit 38. In addition to the regions 92 to 96 similar to the screen D1, the screen D2 includes a region 98 in which candidates for element information that can be changed for the selected element information (hereinafter referred to as “change candidates”) are displayed. The user operates a cursor 99 on the screen D2 via the input unit 25 to select element information to be changed and change candidates. In the example of FIG. 5, the element information of “air bronchogram +” in the region 94 is selected, and the change candidate of “description unnecessary” in the region 98 is selected.

In a case where the element information in the region 94 is selected on the screen D2, the control unit 38 causes the region 98 to display change candidates regarding the selected element information. In a case where the user selects any of the change candidates in the region 98, the control unit 38 changes the selected element information in the region 96 to the selected change candidate.

In a case where some element information among the plurality of pieces of element information generated by the first generation unit 32 is changed, the correction unit 36 corrects only a first sentence corresponding to the element information to be changed, among the plurality of sentences generated by the second generation unit 34. Specifically, the correction unit 36 classifies the plurality of sentences generated by the second generation unit 34 into a first sentence that corresponds to the element information to be changed and a second sentence that does not correspond to the element information to be changed. Furthermore, the correction unit 36 instructs the second generation unit 34 to correct only the first sentence among the first sentence and the second sentence based on the changed element information.

In the example of FIG. 5, the sentence corresponding to the element information of “air bronchogram +” to be changed is the third sentence in group C. The correction unit 36 classifies the sentence in group C as the first sentence that corresponds to the element information to be changed, and classifies the sentences in groups A and B as the second sentences that do not correspond to the element information to be changed. Thereafter, the correction unit 36 instructs the second generation unit 34 to correct only the first sentence in group C, based on the element information changed to “air bronchogram description unnecessary”. The second generation unit 34 generates the sentence again based on the changed element information of group C.

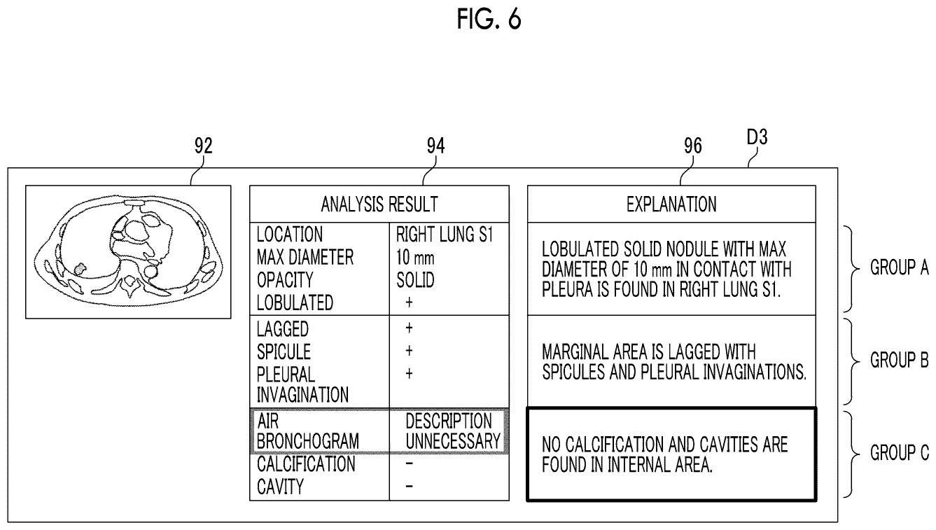

FIG. 6 shows an example of a screen D3 including the corrected first sentence, which is displayed on the display 24 by the control unit 38. As shown in FIG. 6, the control unit 38 may perform control to display, on the display 24, the corrected first sentence (group C), which is corrected based on the changed element information (“air bronchogram description unnecessary”), in an emphasized manner. Further, in the example of FIG. 6, in the region 94, the changed element information (“air bronchogram description unnecessary”) is highlighted. By highlighting only the corrected first sentence in this way, it becomes easier for the user to check which sentence among the plurality of sentences has been corrected.

Note that the above-described classification of the first sentence and the second sentence by the correction unit 36 may be performed in real time while the control unit 38 is receiving changes by the user to the element information. In this case, as shown in FIG. 5, the control unit 38 may perform control to display, on the display 24, the first sentence before correction, which corresponds to the element information to be changed, in an emphasized manner among the plurality of sentences displayed in the region 96. By highlighting the first sentence during the change work in this way, the user can easily check which sentence among the plurality of sentences is affected in the case of changing the element information.

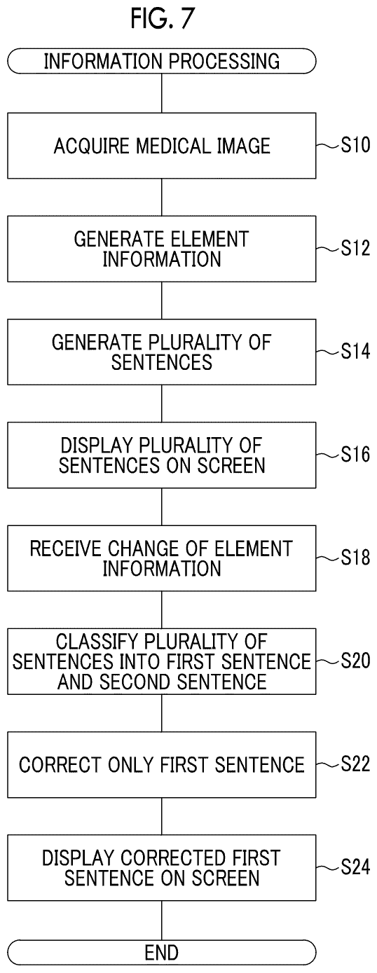

Next, with reference to FIG. 7, operations of the information processing apparatus 10 according to the present exemplary embodiment will be described. In the information processing apparatus 10, as the CPU 21 executes the information processing program 27, information processing shown in FIG. 7 is executed. The information processing is executed, for example, in a case where the user gives an instruction to start execution via the input unit 25.

In Step S10, the acquisition unit 30 acquires a medical image from the image server 5. In Step S12, the first generation unit 32 generates a plurality of pieces of element information based on the medical image acquired in Step S10. In addition, the first generation unit 32 may generate element information based on information input by the user via the input unit 25 and information acquired from an external device. In Step S14, the second generation unit 34 generates a plurality of sentences based on the element information generated in Step S12. In Step S16, the control unit 38 causes the display 24 to display the screen including the plurality of sentences generated in Step S14.

In Step S18, the control unit 38 receives changes made by the user to the element information generated in Step S12. In Step S20, the correction unit 36 classifies the plurality of sentences generated in Step S14 into a first sentence corresponding to the element information to be changed received in Step S18 and a second sentence that does not correspond to the element information to be changed. In Step S22, the correction unit 36 instructs the second generation unit 34 to correct only the first sentence based on the changed element information, and the second generation unit 34 corrects only the first sentence based on the changed element information. In Step S24, the control unit 38 causes the display 24 to display the screen including the first sentence corrected in Step S22, and ends this information processing.

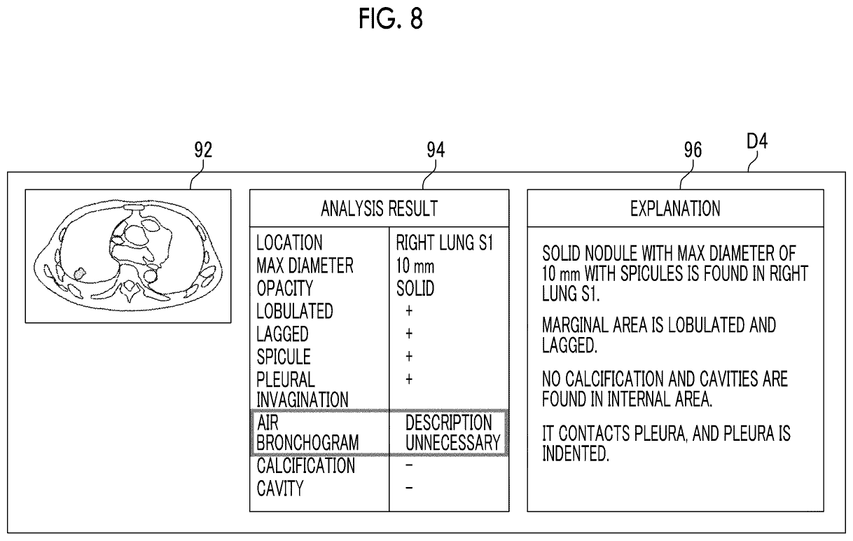

Here, a problem will be described in a case where, among a plurality of sentences, only the first sentence corresponding to the element information to be changed is not corrected. In a case where only the first sentence corresponding to the element information to be changed is not corrected, and in a case where the second generation unit 34 generates a plurality of sentences again based on the changed element information, there are cases where the entirety of the plurality of sentences changes significantly. FIG. 8 shows an example of a screen D4 that may be displayed on the display 24 in a case where the entirety of a plurality of sentences has changed significantly. As shown in FIG. 8, in a case where the entirety of a plurality of sentences is changed, the user may have to check the entirety of the plurality of sentences again, which may be troublesome. In addition, the user may not be able to immediately specify the portion that has been corrected in response to the changed element information, making it difficult to check.

On the other hand, as described above, the information processing apparatus 10 according to one aspect of the present disclosure comprises at least one processor, and the processor is configured to: generate a plurality of sentences based on a plurality of pieces of element information used for diagnosis; and correct, in a case where some element information among the plurality of pieces of element information is changed, only a first sentence corresponding to the element information to be changed among the plurality of sentences. That is, with the information processing apparatus 10 according to the present exemplary embodiment, by correcting only the sentence corresponding to the changed element information, it becomes easier for the user to check the corrected parts, thereby supporting the creation of medical documents.

Further, in the exemplary embodiment described above, the form in which the first generation unit 32 generates the element information has been described, but the present disclosure is not limited thereto. For example, in a case of creating an interpretation report by generating element information in advance by an external device having the same function as the first generation unit 32 that generates element information based on a medical image, the acquisition unit 30 may be configured to acquire element information from the external device.

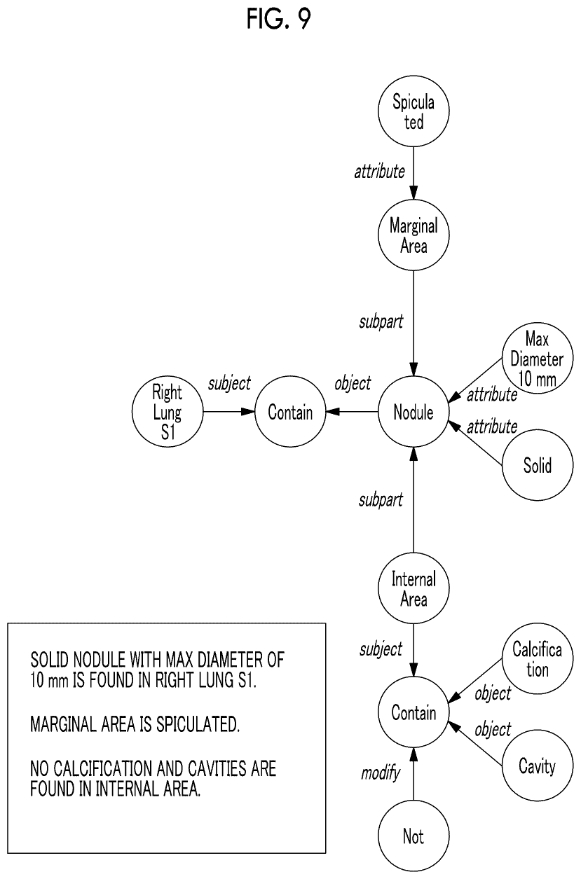

Further, in the exemplary embodiment described above, the form in which the second generation unit 34 generates a plurality of sentences based on element information has been described, but the present disclosure is not limited thereto. For example, a plurality of pieces of element information can be represented by a graph structure represented by a node indicating each of the plurality of pieces of element information and an edge connecting the nodes of the related pieces of element information. FIG. 9 shows an example of a graph structure and a sentence to be generated based on the graph structure. FIG. 9 is a so-called directed graph in which nodes are represented by circles and edges are represented by arrows, and the nodes of the related element information are connected by edges. Also, the meaning of edge is shown in italics.

The second generation unit 34 may generate a plurality of sentences based on a graph structure as shown in FIG. 9. Specifically, the second generation unit 34 generates a graph structure based on the plurality of pieces of element information regarding the medical image generated by the first generation unit 32. Thereafter, the second generation unit 34 generates a plurality of sentences based on the generated graph structure. To generate sentences based on the graph structure, for example, a trained model such as a CNN, which is trained in advance to input a graph structure and output a sentence, may be used. The second generation unit 34 may generate a sentence by inputting the generated graph structure to the trained model.

Further, in the exemplary embodiment described above, the form in which the medical image is used as an example of the image has been described, but the technology of the present disclosure can also use an image other than the medical image as a diagnosis target. For example, the technology of the present disclosure can be applied to the case where a report is created using CT images captured during non-destructive examination of structures, industrial products, pipes, and the like as diagnosis targets.

In the above exemplary embodiment, for example, as hardware structures of processing units that execute various kinds of processing, such as the acquisition unit 30, the first generation unit 32, the second generation unit 34, the correction unit 36, and the control unit 38, various processors shown below can be used. As described above, the various processors include a programmable logic device (PLD) as a processor of which the circuit configuration can be changed after manufacture, such as a field-programmable gate array (FPGA), a dedicated electrical circuit as a processor having a dedicated circuit configuration for executing specific processing such as an application-specific integrated circuit (ASIC), and the like, in addition to the CPU as a general-purpose processor that functions as various processing units by executing software (program).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. Second, as represented by a system-on-chip (SoC) or the like, there is a form of using a processor for realizing the function of the entire system including a plurality of processing units with one integrated circuit (IC) chip. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

In the above exemplary embodiment, the information processing program 27 is described as being stored (installed) in the storage unit 22 in advance; however, the present disclosure is not limited thereto. The information processing program 27 may be provided in a form recorded in a recording medium such as a compact disc read-only memory (CD-ROM), a digital versatile disc read-only memory (DVD-ROM), and a universal serial bus (USB) memory. In addition, the information processing program 27 may be configured to be downloaded from an external device via a network. Further, the technology of the present disclosure extends to a storage medium for storing the information processing program non-transitorily in addition to the information processing program.

The technology of the present disclosure can be appropriately combined with the above-described exemplary embodiment. The described contents and illustrated contents shown above are detailed descriptions of the parts related to the technology of the present disclosure, and are merely an example of the technology of the present disclosure. For example, the above description of the configuration, function, operation, and effect is an example of the configuration, function, operation, and effect of the parts related to the technology of the present disclosure. Therefore, needless to say, unnecessary parts may be deleted, new elements may be added, or replacements may be made to the described contents and illustrated contents shown above within a range that does not deviate from the gist of the technology of the present disclosure.

The disclosure of JP2021-162034 filed on Sep. 30, 2021 is incorporated herein by reference in its entirety. All documents, patent applications, and technical standards described in the present specification are incorporated in the present specification by reference to the same extent as in a case where each of the documents, patent applications, technical standards are specifically and individually indicated to be incorporated by reference.

What is claimed is:

1. An information processing apparatus comprising at least one processor, wherein the processor is configured to:
generate a plurality of sentences based on a plurality of pieces of element information used for diagnosis; and
correct, in a case where some element information among the plurality of pieces of element information is changed, only a first sentence corresponding to the element information to be changed among the plurality of sentences.

2. The information processing apparatus according to claim 1, wherein the processor is configured to:
classify the plurality of sentences into the first sentence and a second sentence that does not correspond to the element information to be changed; and
correct only the first sentence among the first sentence and the second sentence based on the changed element information.

3. The information processing apparatus according to claim 1, wherein the element information is information indicating at least one of a name, a property, a measured value, a position, or an estimated disease name related to a region of interest included in a medical image, or an imaging method, an imaging condition, or an imaging date and time related to imaging of the medical image.

4. The information processing apparatus according to claim 3, wherein the region of interest is at least one of a region of a structure included in the medical image or a region of an abnormal shadow included in the medical image.

5. The information processing apparatus according to claim 1, wherein the processor is configured to perform control to display the plurality of sentences on a display device.

6. The information processing apparatus according to claim 5, wherein the processor is configured to perform control to display the plurality of sentences on the display device by grouping the plurality of sentences based on the element information to which the sentences correspond.

7. The information processing apparatus according to claim 5, wherein the processor is configured to perform control to display the corrected first sentence in an emphasized manner on the display device.

8. The information processing apparatus according to claim 5, wherein the processor is configured to, in a case of receiving a change in the element information, perform control to display the first sentence before correction in an emphasized manner on the display device.

9. The information processing apparatus according to claim 1, wherein the processor is configured to:
acquire a medical image; and
generate the element information based on the acquired medical image.

10. The information processing apparatus according to claim 1, further comprising an input unit, wherein the processor is configured to generate the element information based on information input via the input unit.

11. The information processing apparatus according to claim 1, wherein the processor is configured to acquire the element information from an external device.

12. An information processing method comprising:
generating a plurality of sentences based on a plurality of pieces of element information used for diagnosis; and
correcting, in a case where some element information among the plurality of pieces of element information is changed, only a first sentence corresponding to the element information to be changed among the plurality of sentences.

13. A non-transitory computer-readable storage medium storing an information processing program causing a computer to execute:
generating a plurality of sentences based on a plurality of pieces of element information used for diagnosis; and
correcting, in a case where some element information among the plurality of pieces of element information is changed, only a first sentence corresponding to the element information to be changed among the plurality of sentences.
